(19) United States
(12) Patent Application Publication
(10) Pub. No.: US 2021/0015393 A1
(43) Pub. Date: Jan. 21, 2021

(54) EEG HEADGEAR ASSEMBLY WITH ELECTRODE POSITIONING SYSTEM

(71) Applicant: JORDAN NEUROSCIENCE, INC., Redlands, CA (US)

(72) Inventors: Kenneth George Jordan, Riverside, CA (US); Gary Reasoner, Newport Beach, CA (US)

(21) Appl. No.: 16/981,630

(22) PCT Filed: Mar. 18, 2019
(86) PCT No.: PCT/US2019/022828

§ 371 Date: Sep. 16, 2020

Related U.S. Application Data
(60) Provisional application No. 62/644,346, filed on Mar. 16, 2018.

(51) Int. Cl. A61B 5/0478; A61B 5/00

(52) U.S. Cl. CPC A61B 5/0478; A61B 5/6803; A61B 2562/04; A61B 5/72; A61B 2562/0217; A61B 5/0006

(57) ABSTRACT

An EEG headgear assembly for placement on the head of a subject which includes headgear formed from elastic straps and wired electrodes. The electrodes are secured to the headgear by means of elastic pockets which press an electrode head toward a subjects skin.

### BACKGROUND

Electrical potentials are generated by ionic current flows between the neurons of the brain. Such current flows can be detected by electroencephalography (EEG). EEG devices record the brain's electrical activity from the scalp and display it in the form of an electroencephalogram (EEG), which generally depicts waveforms of varying frequency measured in Hertz (cycles per second) and amplitude as measured in voltage. EEG waveforms are generally classified according to their frequency (standard frequency ranges termed alpha, beta, theta, and delta), amplitude, and shape, as well as by the locations on the scalp at which they are recorded.

An irregular brain wave pattern can be indicative of a particular brain pathology. Distinctive EEG patterns are seen, for example, in acquired brain injury (ABI), accelerated cerebral edema, expanding intracranial masses, and severe cerebral ischemia. Such patterns include encephalographic seizures, markedly slow frequencies, amplitude suppression, “burst suppression,” and periodic epileptiform discharges (PEDS). While not specific for particular diagnoses, these patterns provide an early, sensitive and reliable warning of severe or worsening brain damage.

The use of EEG as soon as possible after suspected brain damage is of particular importance because EEG can detect brain injuries at an early stage, before they progress to a more advanced or irreversible degree. This is called “Point-of Injury” (POI) EEG or “Triage EEG.” For example, in moderate to severe traumatic ABI, such as from the blast of an explosion or a concussive impact, or in non-traumatic ABI from a stroke or hemorrhage, death or irreversible brain injury are often due to “secondary progression” events that produce clear EEG abnormalities including brain edema, enlarging hemorrhages, seizures, torn or occluded blood vessels, and brain herniation. In the context of acute traumatic brain injury (ATBI), EEG can provide an early warning of worsening brain damage and provide clues about the underlying disease process.

Conventional EEG set-up methods are labor intensive, and the technical expertise needed to prepare a patient and apply EEG electrodes properly may not be readily available. The International 10-20 System of EEG electrode placement, for example, involves positioning 20-24 electrodes on a patient's scalp, which may take up to an hour. Delays of hours may occur before a trained EEG technologist “on call” arrives, an EEG procedure is performed, and the results interpreted. Such delays can result in long term or irreversible brain damage. Patient's with ABIs, such as those in a coma in a hospital's Emergency Department, or with seizures in a paramedic ambulance, or with a concussion in a sports venue need emergency EEG as soon as possible, preferably at the point of injury.

EEG headgear is available with pre-arranged electrode placement locations, such as the product sold as the BRAINET EEG template (available from Jordan Neuroscience, Inc.). Such headgear act as templates for the placement of electrodes, and can reduce the amount of time needed to properly apply electrodes to a patient. There remains a need, however, for an improved EEG headgear and electrode positioning system for emergency EEG in ABI that meets both patient and commercial needs.

### SUMMARY

The present EEG headgear (1) generally comprises a piece of EEG headgear or (or template, 10) for applying electrodes, wires (40), and electrodes (50). The headgear (10) has a top end (11), bottom end (12), front side (13), back side (14), right side (15), and left side (16), and preferably comprises a plurality of straps (20) along which the electrodes are positioned. The straps (20) have an upper side (21) and a lower side (22), and preferably include a transverse strap (26), a coronal strap (28), and a sagittal strap (30). The coronal strap (28) is preferably secured around a subject's chin when in use. In some embodiments, the distal ends of the coronal strap (28) can be connected by a chin strap (24), which preferably includes a slit (23) to accommodate a chin. A pair of bias straps (25) preferably attach to the distal ends of the coronal strap (28), so that each bias strap (25) connects a respective distal end to the back side (14) of the headgear (10), either to the transverse strap (26) or the sagittal strap (30).

The present EEG headgear (1) further comprises wires (40) having a proximal end (41) in electrical communication with an electrical connector (60), such as an electrical jack, which can be attached to a mating electrical connector of a transmitter (150) or can be placed directly in communication with processing and recording circuitry. The distal end (42) of each of the wires (40) is attached to and in electrical communication with an electrode (50), such as through a connector (57). Preferably, each electrode (50) has an upper side (52) and lower side (54), and the lower side preferably comprises a cup (55) for retaining a conductive gel or other material for facilitating transmission of an electrical signal from the subject's scalp to the electrode (50).

The electrode (50) is positioned on the headgear through the use of a pocket (70) formed by an elastic panel (80) placed on the upper side (21) of a strap (20). The elastic panel (80) preferably comprises a proximal side (81) at the proximal opening (75) of the pocket, a distal side (82), right side (83), left side (84), upper side (85), and lower side (86). The lower side (86) of the panel (80) faces the upper side (21) of the strap (20) and forms an upper side (72) of the pocket (70), while the upper side (21) of the strap (20) forms the lower side (74) of the pocket. The pocket (70) can be formed by sewing the distal side (82), right side (83), and left side (84) of the panel (80) with thread (90) to secure it to the strap (20). The lower side (74) of the pocket (70) further comprises a strap opening (76) through which the lower side (54) of the electrode (50) can be extended in order to place the lower side (54) of the electrode (50) in contact with the skin of a subject.

### FIGURES

FIG. 1 is a front elevation view of an embodiment of the present EEG headgear assembly placed on a subject's head, including a transmitter module;

FIG. 2 is a top plan view thereof;

FIG. 3 is a front elevation view of another embodiment of the present EEG headgear assembly, without a transmitter module;

FIG. 4 is a top plan view thereof;

FIG. 5 is a right-side elevation view thereof;

FIG. 6 is a left-side elevation view thereof;

FIG. 7 is a top plan view of an embodiment of a pocket of the present EEG headgear, with an electrode head positioned in the pocket;

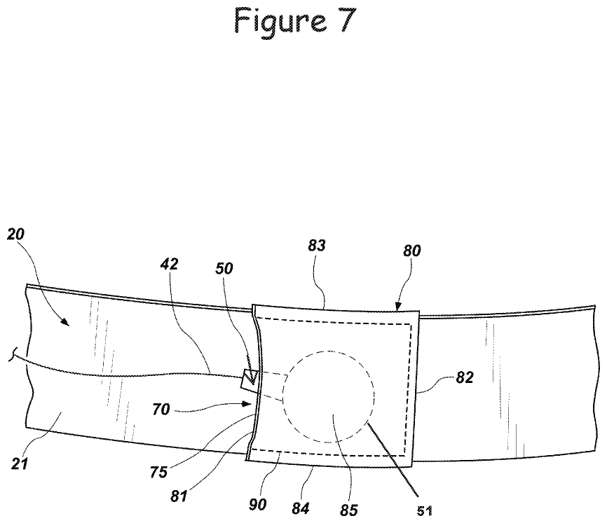

FIG. 8 is a bottom plan view thereof with a cup electrode;

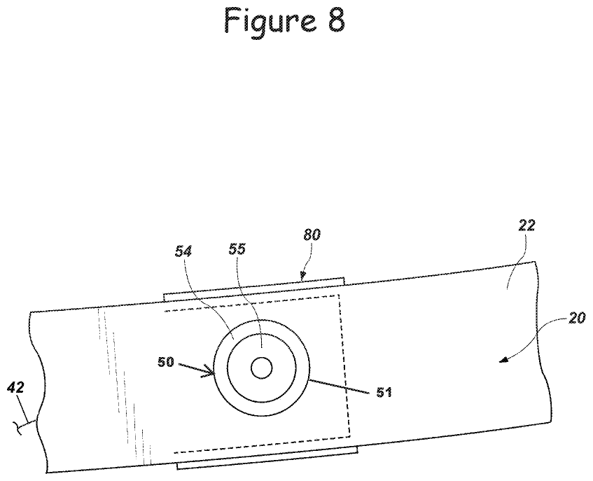

FIG. 9 is a bottom plan view thereof with a disc electrode;

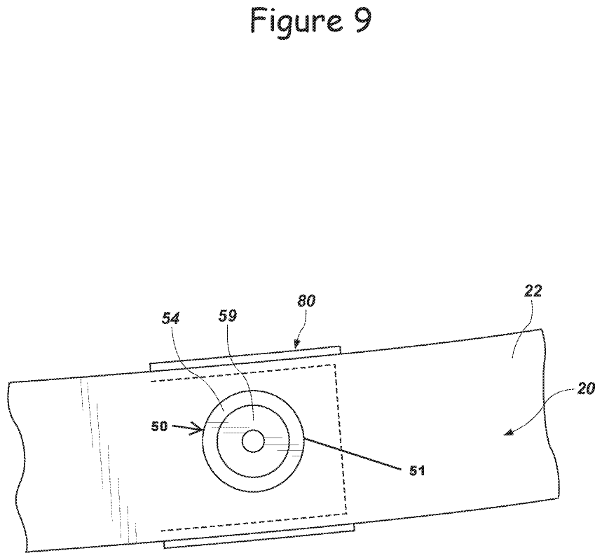

FIG. 10 is a bottom plan view of the pocket of FIG. 6, with the electrode removed from the pocket;

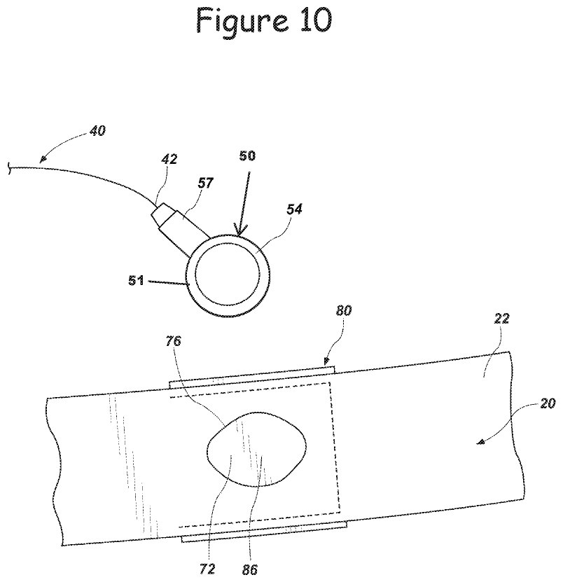

FIG. 11 is a top plan view of the pocket of FIG. 6, with the electrode fully removed from the pocket; and

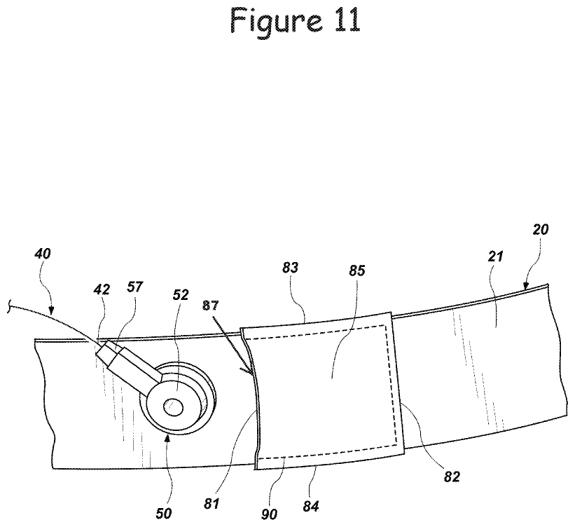

FIG. 12 is a top plan view of the pocket of FIG. 6, with the electrode partially inserted in the pocket.

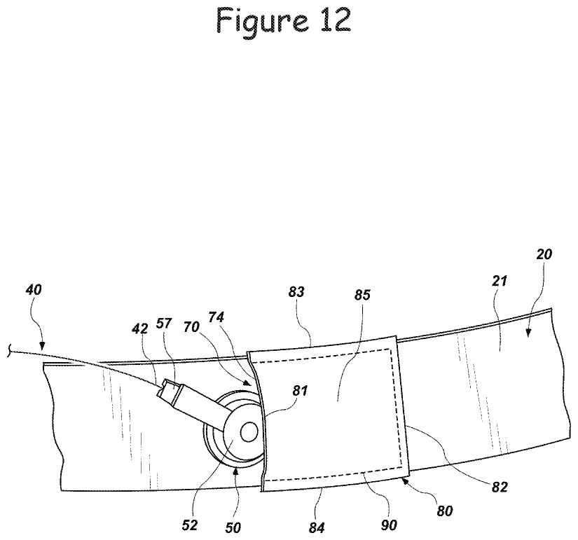

The reference numbers in the figures have the following meanings:

| Component | Reference Number |
|---|---|
| EEG headgear assembly | 1 |
| Headgear | 10 |
| Headgear top | 11 |
| Headgear bottom | 12 |
| Headgear front | 13 |
| Headgear back | 14 |
| Headgear right side | 15 |
| Headgear left side | 16 |
| Straps | 20 |
| Strap upper side | 21 |
| Strap lower side | 22 |
| Chin strap slit | 23 |
| Chin strap | 24 |
| Bias strap | 25 |
| Transverse strap | 26 |
| Coronal strap | 28 |
| Strap material | 29 |
| Sagittal strap | 30 |
| Wires | 40 |
| Proximal end | 41 |
| Distal end | 42 |
| Electrode | 50 |
| Electrode head | 51 |
| Electrode upper side | 52 |
| Electrode lower side | 54 |
| Electrode cup | 55 |
| Electrode connector | 57 |
| Disc electrode | 59 |
| Electrical connector | 60 |
| Pocket | 70 |
| Pocket upper side | 72 |
| Pocket lower side | 74 |
| Pocket proximal opening | 75 |
| Pocket lower opening | 76 |
| Elastic panel | 80 |
| Panel proximal side | 81 |
| Panel distal side | 82 |
| Panel right side | 83 |
| Panel left side | 84 |
| Panel upper side | 85 |
| Panel lower side | 86 |
| Receptacle | 87 |
| Thread | 90 |
| Transmitter module | 150 |
| Transmitter | 151 |
| Container | 155 |
| Container window | 153 |

### DESCRIPTION

### Definitions

As used herein, the following terms and variations thereof have the meanings given below, unless a different meaning is clearly intended by the context in which such term is used.

“About” and “approximately” refer to a quantity or distance within 10% of the referenced quantity, distance, or location unless the circumstances of such usage would indicate a different meaning.

“Coronal” refers to an orientation that is generally within or parallel to a subject's coronal plane, which is a hypothetical vertical plane that divides a subject's body (or a portion thereof) into ventral (front) and dorsal (back) sections, preferably of approximately equal sizes (by width or volume). “Coronal” as used herein may refer to the orientation of components of the present device which are oriented generally in the coronal axis of a subject when the present device is worn by the subject, but which may deviate therefrom by up to 30% or so, for example to accommodate placement of strap portions between the crown and chin of a subject.

“Crown” refers to the top portion of a subject's head when the subject is standing upright, approximately where the sagittal and coronal planes of the subject intersect.

“EEG” refers to electroencephalography, i.e. the recording of electrical activity at a subject's scalp, in order to measure voltage fluctuations resulting from ionic current flows within the neurons of the subject's brain.

“Elastic” refers to a material which is reversibly deformable, such that the material can be placed under stress and can be bent, elongated, or compressed and thereby be changed from a first conformation (such as a first length or shape) to a second conformation, and which will return to the first conformation after the stress is removed.

“Headgear” refers to an item worn on a subject's head. In the context of the present application, headgear is formed from an elastic material, preferably elastic straps assembled to allow them to fit a subject's head and overlay locations useful for obtaining EEG signals.

“Horizontal,” with respect to the present appliance, refers to disposition in a plane which is generally parallel to the transverse plane of a subject, i.e. within 30 degrees of such a parallel plane.

“Jack” refers to an electrical connector comprising a “female” electrical contact or socket, i.e. which receives another electrical connector, and is generally the more “fixed” connector of a connector pair comprising a jack and a plug.

“Panel” refers to a section or piece of material extending over or covering a predetermined area. Panels are generally flat, i.e. are relatively thin as compared to the extent of their length or width and can be curved or planar. Materials used to form the panels used in the present invention are flexible, such as fabric.

“Plug” refers to an electrical connector comprising a “male” electrical contact or pin, i.e. which is inserted into or onto another electrical connector and is generally the more movable (less fixed) connector of a connector pair comprising a jack and a plug.

“Receptacle” refers to a portion or component of the present device able to receive and retain another portion or component of the device, or to receive and retain an external component or device.

“Sagittal” refers to an orientation that is within or parallel to a subject's sagittal plane, i.e. a hypothetical vertical plane which passes from ventral (front) to dorsal (back) of a subject's body, dividing the body into approximately equally sized right and left halves. “Sagittal” as used herein may refer to the orientation of components of the present device which are oriented generally in the sagittal axis of a subject but which may deviate therefrom by up to 30% or so when the present device is worn by the subject.

“Scalp” refers to the skin covering the head.

“Sew” refers to the attachment of a thread or other length of material to a cloth by passing the thread through one side of the cloth and then back through the other side a plurality of times.

“Strap” refers to a length of flexible material. Straps in the present EEG headgear are used to secure one or more component parts of the present EEG headgear to a subject's head.

“Thread” refers to a strand or length of material with a diameter or thickness significantly less than its length, and generally less than 5 millimeters in diameter.

“Transverse” refers to an orientation that is within or parallel to a hypothetical horizontal plane that divides a subject's body into superior (upper) and inferior (lower) parts and is generally perpendicular with respect to the sagittal and/or coronal planes of the subject. “Transverse” as used herein may refer to the orientation of components of the present device which are oriented generally in the transverse axis of a subject but which may deviate therefrom by up to 30% or so when the present device is worn by the subject.

“Vertical,” with respect to the present appliance, refers to disposition in a plane which is generally parallel to the sagittal and/or the coronal plane of a subject, i.e. within 30 degrees of such a parallel plane.

Terms of relative position such as “upper,” “lower,” “above,” “below,” “top”, “bottom,” “front,” “rear,” “right,” “left,” and similar terms will be used to designate areas and positions of portions of the components of the present device with respect to other portions of the present device, but it is to be understood that these terms are relative and are not absolute terms. For example, “right” and “left” will thus be used to designate opposing lateral positions but will be understood to be relative terms.

The term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise. Ranges which are described as being “between” two values include the indicated values.

### EEG Headgear Assembly

Headgear and Straps

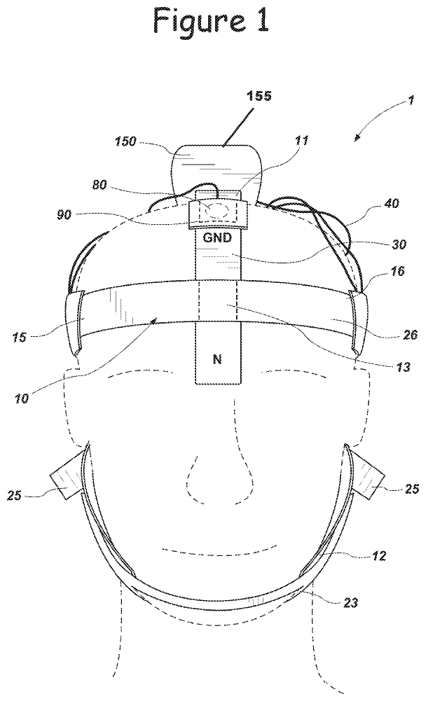

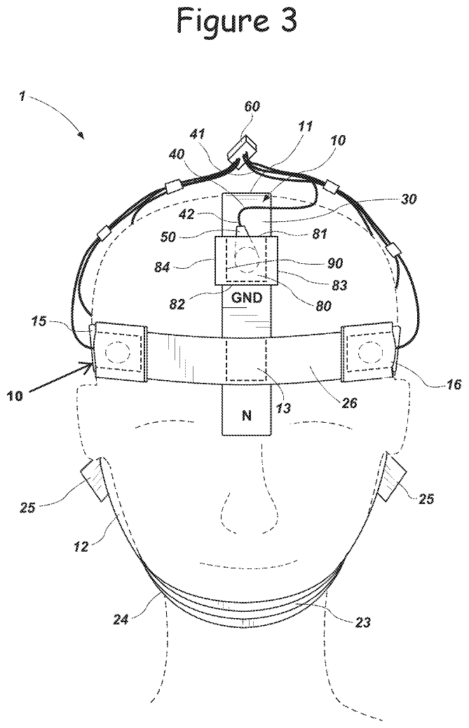

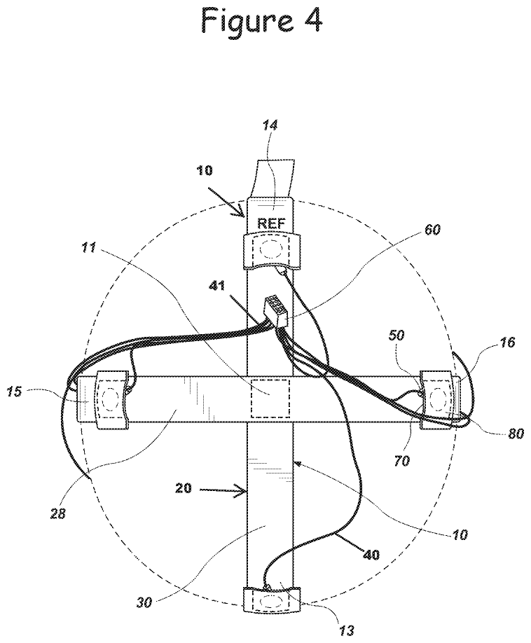

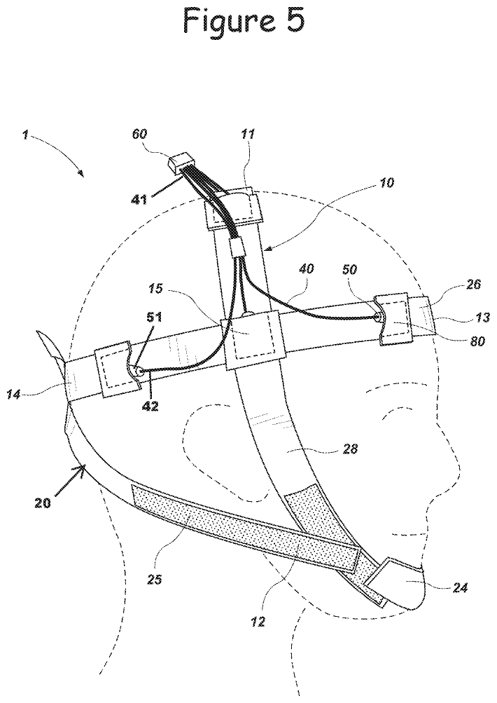

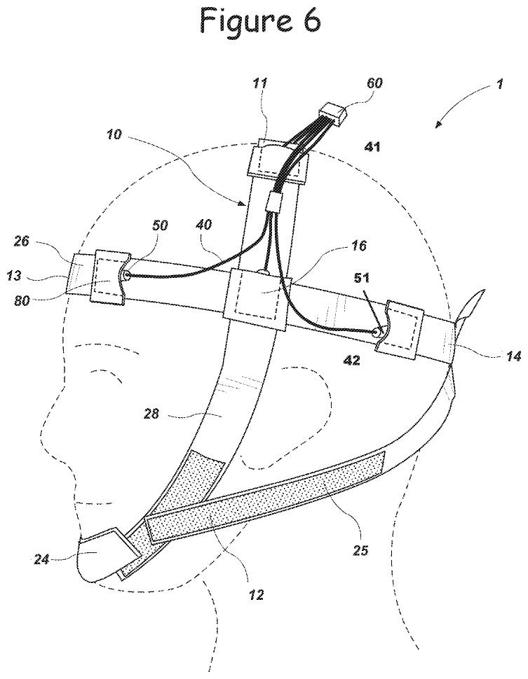

The present EEG headgear assembly 1 generally comprises a headgear 10 for applying electrodes, wires 40, and electrodes 50. As shown in FIGS. 1-6, the headgear 10 has a top end 11, bottom end 12, front side 13, back side 14, right side 15, and left side 16, and preferably comprises a plurality of straps 20 along which the electrodes are positioned. One of skill in the art will appreciate that a greater or lesser number of straps or strap portions than those used in the illustrated embodiments can be used. The straps 20 can be composed of a plurality of flat, longitudinally extending, preferably elastic pieces of material, or alternatively can be formed as a unitary body from a single sheet of material or by plastic casting. The straps are arranged so as to be allow the headgear 10 to be retained on a subject's head, and to cover areas where electrode heads 51 need to be placed in order to conduct an EEG procedure.

The headgear 10 is preferably formed from a plurality of straps 20, each of which has an upper side 21 and a lower side 22. The straps or strap portions of a unitary construction preferably include at least a transverse strap 26, a coronal strap 28, and a sagittal strap 30. The sagittal strap 30 is adapted to be placed approximately in the sagittal plane of the subject's head from a front end 13 on the subject's forehead to a rear end 14 at the back of the subject's head. The coronal strap 28 is placed on the subject's head in an orientation that is generally parallel to the coronal plane of the subject's body, while the transverse strap 26 is placed on the subject's head in an orientation that is generally parallel to a transverse plane. Placement of the straps 26, 28, and 30 may deviate from the coronal, sagittal, or transverse orientations in order to appropriately place electrodes carried by the straps 20, conform to the contours of the subject's head, and/or better secure the headgear 10. Deviations in the orientation of the straps may range from 10°-20° to up to 45°.

The headgear 10 can be secured to a subject's chin in various ways known to the art, such as with fasteners attached to the distal ends of the coronal strap 28. In the illustrated embodiments, each end of a chin strap 24 is attached to a respective end of the coronal strap 28 using a pair of mated connectors, preferably hook and loop fasteners (such as VELCRO brand fastener strips). The fastener strap 24 preferably includes a slit 23 through the length of the chin strap 24, in order to better anchor the strap to a patient's chin.

Bias straps 25 can also be added to the headgear 10 to further secure it in place. A preferred construction for the bias straps 25 connects a left bias strap and a right bias strap to the portion of the sagittal strap 30 located on the back end 14 of the headgear 10. The bias straps 25 are each preferably removably connected to the coronal strap 28 on the right side 15 and left side 16 of the headgear 10, respectively. The removable connection can be, for example, a fastener such as a hook and loop fastener.

The positions for electrodes 50 on the headgear 10 can be determined according to the standard 10-20 electrode placement system for locating EEG electrodes, or according to other standards used in the art. In the standard 10-20 system, various numbers of electrodes can be placed at specifically measured scalp locations. Typically, from 8 to 24 electrodes are placed on a subject's head at positions which are commonly labeled Fp1, Fp2, Fpz, F7, F3, Fz, F4, F8, A1, T7, C3, Cz, C4, T8, A2, P7, P3, Pz, P4, P8, O1, Oz and O2. However, other electrode configurations are possible, some with fewer than 8 electrodes, as is known to the art.

In an alternative embodiment, the headgear 10 can contain no openings for the F3/F4 pair of electrodes or the P3/P4 pair of electrodes, and the openings for the Fz and Pz electrodes can be eliminated. In this embodiment, the headgear 10 has an opening geometry consisting essentially of thirteen strap openings 76 located according to the International 10-20 System specification for positioning electrodes at points Fp1, Fp2, F7, F8, T3, T4, Cz, C3, C4, T5, T6, O1, and O2, and a fourteenth opening for the ground 71, which is preferably positioned 25% of the distance from a hypothetical line that circumscribes the subject's head in approximately a transverse plane toward a line which the vertically traverses the subject's head in approximately a coronal plane. This location for the ground is approximately 20% of the distance from the nasion to the inion and can be varied by approximately half a centimeter in any direction. A reference electrode (REF) can also be provided.

The headgear 10, in particular the straps 20, can preferably be made from an elastic material, in particular an elastic cloth, in order to secure the headgear 10 to the head of a subject as well as to secure the electrodes 50 in the proper position. In a preferred embodiment, the elastic cloth is formed from a polyurethane or from a polyether-polyurea copolymer, such as the elastic material sold as spandex. In this case the headgear 10 is preferably sized such that the sagittal strap 30 and the transverse strap 26, prior to being stretched, have a diameter which is less than the diameter of the portion of the subject's head which they are designed to cover when the subject is fitted with the headgear 10. The diameter of the subject's head should be within the elastic limit of the material forming the sagittal strap 30 and the transverse strap 26, so that the headgear 10 can be stretched to fit comfortably over the subject's head and then to contract so as to hold the headgear 10 in a predetermined position by a friction fit.

Electrical Components

The present EEG headgear assembly 1 further comprises a plurality of wires 40, each having a proximal end 41 in electrical communication with an electrical connector 60 and a distal end 42 in electrical communication with an electrode 50. The proximal end 41 of each wire is preferably placed into electrical communication with an amplifier and/or with other circuitry needed to process and/or display an electrical signal received through the electrodes 50 via the electrical connector 60 during an EEG procedure. Such circuitry can include a filter, and in the case of digital EEG devices, can include an analog-to-digital converter. In one embodiment, the proximal ends 41 of wires 40 are placed in direct electrical connection with circuitry needed to process and/or display electrical signals received through the electrodes 50 by connecting the wires 40 to other wires extending between the electrical connector 60 and such circuitry.

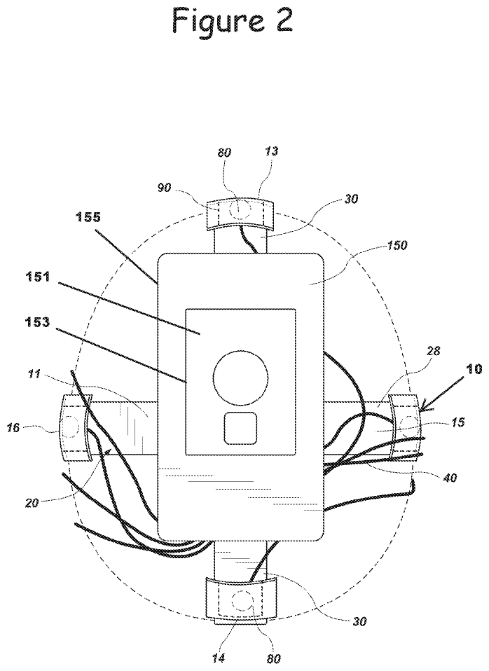

In a preferred embodiment, the wires 40 are placed in electrical communication with a wireless transmitter module 150, which transmits electrical signals received from each of the electrodes 40 to another device, for example to a device capable displaying and/or recording electrical activity from the brain of a subject wearing the present EEG headgear assembly 1. Any of a number of transmitters 151 known to the art can be used, for example a transmitter such as that described in U.S. Pat. No. 10,130,278, and can make use of wireless protocols such Bluetooth or WiFi. The transmitters 151 of the transmitter module 150 can also comprise additional circuitry and functionalities, such as signal processing capabilities and electrode impedance indicators for example. As shown in FIG. 2, the upper side of the container 155 preferably includes a transparent window 153, preferably covered with a transparent plastic material, in order to allow transmitter controls, visual indicators, and/or other elements of the transmitter 151 to be seen.

In the embodiment shown in FIGS. 1 and 2, the transmitter 151 is retained in a container 155 having a lower side which can be reversibly secured to the upper side sagittal strap 30 and/or to the coronal strap 28, such as with VELCRO or other hook and loop fasteners or with other fasteners known to the art such as buttons or snap fasteners. While the container 155 and transmitter 151 are intended for use with multiple subjects, i.e. are not disposable, the remainder of the EEG headgear assembly 1, namely the headgear 10 and electrodes 50, can be disposed of after use by a single individual, i.e. can be disposable, which may be desirable for hygienic reasons. The removability of the transmitter module 150 from the remainder of the EEG headgear assembly 1 also allows the module 150 to be detached for charging when needed.

The electrical connector 60 facilitates connection of the proximal ends 41 of the wires 40 to such circuitry. The electrical connector 60 can be, for example, an electrical jack such as a DIN connector. The illustrated embodiments show a 10-pin DIN connector, but other connectors can be used with greater or fewer pins corresponding to the number of recording electrodes. The transmitter 150 or other electrical component of the EEG device is provided with a mating electrical connector, for example a plug (not shown).

Electrodes

The distal end 42 of each of the wires 40 is attached to and in electrical communication with an electrode head 51, such as through a connector 57. Preferably, each electrode head 51 has an upper side 52 and lower side 54, and the lower side preferably comprises a cup 55 or other appropriate surface for retaining a conductive gel or other material for facilitating transmission of an electrical signal from the subject's scalp to the electrode head 51.

Although a variety of electrodes known for use with EEG can be used with the present EEG headgear assembly 1, such as needle electrodes, in a preferred embodiment the electrodes 50 comprise disc (cup) electrodes 55. Needle electrodes have the disadvantage of requiring that the subject's skin be pierced by the electrode. Cup electrodes avoid such issues, as they do not require skin abrasion, although abrasion can be used to facilitate electrical signal transmission. Other types of electrodes which can be used include, for example, flat disc electrodes 59, such as disc electrodes with a mesh design to hold a conductive medium, dry electrodes which do not use a conductive paste or other medium, and press-on electrodes such as the PRESSON electrode sold by Rhythmlink (Columbia, S.C.).

Cup electrodes can be made from a variety of conductive materials, including tin, silver, gold, steel, or ABS plastic coated with Ag/AgCl. A preferred electrode is a conductive plastic cup electrode, for example an electrode formed from silver-silver/chloride impregnated plastic (available from Ives EEG, Inc., Newburyport, Mass.) which are transparent to CT scans. They typically have a diameter in the range of 4-10 mm. Preferably, a conductive liquid, gel, or solid can be placed in the lower side 54 of the cup 55 of the electrode 50 or directly on a subject's skin in order to enhance transmission of an electrical signal from a subject. The conductive material can be an electrolyte solution, emulsion, paste, or other conductive liquid, or can be a hydrogel, solid gel, or other conductive gel material.

Pocket Fasteners

In the present EEG headgear assembly 1, the electrode heads 51 are positioned in the headgear 10 through the use of pocket fasteners 70 located on the straps 20 of the headgear 10. The pocket fasteners (“pockets”) 70 comprise an elastic panel 80 secured to the upper side 21 of a strap 20. A distal side 82, right side 83, and left side 84 of the panel are preferably all secured to the strap 20, the while proximal side 81 of the elastic panel 80 is not secured, so as to form a proximal opening 75 of the pocket through which an electrode head 51 can be placed into the pocket 70. The sides of the panel 80 can be secured to the upper side 21 of a strap in ways known to the art. For example, the right, left, and distal sides of the panel 80 can be sewn with thread 90 in order to secure them to the strap 20. By securing at least the right and left sides of the panel 80 to the strap 20 in this way, a receptacle 87, i.e. a chamber or space, can be created between the upper side 21 of the strap 20 and the lower side 86 of the panel 80 for retaining an electrode head 51 within the pocket 70. The lower side 86 of the panel 80 thereby forms the upper side 72 of the pocket 70, and the upper side 21 of the strap forms the lower side 74 of the pocket 70.

The lower side 74 of the pocket 70 further comprises a strap opening 76 through which the lower side 54 of the electrode head 51 can be extended in order to place the lower side 54 of the electrode head 51 in contact with the skin of a subject, or through which the lower side 54 of the electrode head 51can at least make contact with the skin of a subject. The strap opening 76 is or comprises an opening in the portion of the strap 20 above which the elastic panel 80 is attached. The elastic panel 80 overlays the strap opening 76, so that when an electrode head 51 is retained within the strap opening 76, the upper side 72 of the pocket 70 (i.e., the surface of the lower side 86 of the panel 80) contacts the upper side 52 of the electrode head 51.

The electrode head 51 requires no physical or structural attachment to the pocket 70, as the physical properties and spatial relationship of the elastic pocket panel 80 and the subjacent portion of the elastic strap 20 securely hold the electrode head 51 in position. In a non-tensioned state, the elastic panel 80 and the portion of the strap 20 to which it is attached are in contact or are closely adjacent, such that the distance between the lower side 74 and upper side 72 of the pocket 70 is less than the height of the electrode between the upper side 52 and lower side 54 of the electrode head 51. Because the elastic panel 80 is elastic, when it is reversibly deformed by insertion of the electrode head 51 between the lower side 74 and upper side 72 of the pocket, the elastic panel 80 is stretched and placed in tension and exerts a force on the upper side 52 of the electrode 50 due to such tension. The elastic panel 80 thereby exerts a downward pressure on the electrode head 51, i.e. toward the strap 20 and in the direction of a subject's skin when in use, thereby securing the electrode on a subject's skin and within the pocket 70.

The elastic panels 80 can be formed in any of a number of ways known to the art, such as by weaving or knitting elastic fibers, or by polymerizing elastic materials in the form of a panel. In a preferred embodiment, the elastic cloth is formed from fibers made of polyurethane or a polyether-polyurea copolymer, such as the elastic material generally referred to as spandex, LYCRA™ or elastane. One preferred material useable in the present article is CLEERSPAN™ polyurethane thread, available from Radicispandex Corp. (Tuscaloosa, Ala.).

The pocket fasteners 70 are preferably identified with predetermined colors or other indicia in order to allow easy identification. Preferably, the indicia or color of a pocket 70 is matched by the indicia or color of the corresponding electrode or electrode wire of the electrode which should be fastened to it. This assists a less technically trained individual in determining the proper location of the electrodes and replacing the electrodes in the right location if removed.

The present headgear assembly 1 can be used to perform EEG by placing the headgear 10 onto the head of a subject. Advantageously, strap openings 76 are provided only where electrode heads 51 need to be placed in order to perform EEG on the subject, so that after the headgear assembly 1 is positioned on the subject's head, the electrode heads 51 of the electrodes 50 can be placed into the pocket fasteners 70 by sliding them through the proximal openings 75 and into the receptacles 87 of the pockets 70. An EEG procedure can then be performed by placing the electrodes 50 in electrical communication with the appropriate equipment for detecting and preferably recording electrical activity in the subject's brain.

The pocket fasteners 70 provide numerous advantages for securing electrodes in a piece of EEG headgear 10. These fasteners allow attachment of electrodes with only a minimum of training, as insertion of electrodes through the proximal openings 75 of the pockets 70 is a simple matter. Likewise, each electrode head 51 can be easily removed from the headgear assembly 1 by a gentle tug that removes it from the pocket 70 through the opening 75. The electrode head 51 can then be replaced or re-inserted into the pocket 70. This is important if an electrode needs to be replaced, cleansed or otherwise serviced for an adequate recording. The present pocket fasteners include no locking mechanisms, and no additional steps are required to establish and maintain a secure and stable positioning of the electrodes 50 on the headgear 10 other than insertion into the pocket 70.

In addition, the pocket fastener design allows the electrodes 50 to be inserted into the pockets 70 in at least three different ways. They can be inserted in the pockets 70 at the factory and therefore be pre-positioned on the EEG headgear assembly 1 for commercial distribution and storage, ready for application on a subject's head. The electrodes can also be manually inserted into the pockets 70 just before the headgear assembly 1 is applied to the subject's head. Alternatively, the electrodes 50 can be inserted into the pockets 70 after the headgear assembly 1 has been applied to the subject's head.

Manufacture of the pocket fasteners also requires only minimal skill, as they can be formed by sewing, and the pockets 70 can be made from inexpensive materials. The pocket fasteners 80 are also soft, and so present no hard surfaces that might potentially injure a subject using the headgear assembly 1 or a caregiver interacting with the assembly. The elastic panels 80 additionally provide an electrically insulating layer over an electrode head 51 in the pocket 70, thereby enhancing EEG signal integrity.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

Recitation of value ranges herein is merely intended to serve as a shorthand method for referring individually to each separate value falling within the range. Unless otherwise indicated herein, each individual value is incorporated into the specification as if it were individually recited herein. All references cited herein are incorporated by reference in their entirety.

1. A headgear assembly for performing electroencephalography (EEG), comprising:
(a) EEG headgear for retaining a plurality of electrodes on the head of a subject, the headgear comprising one or more elastic straps each having an upper side and a lower side, wherein the one or more elastic straps include a coronal strap portion extending coronally around a subject's head between the crown and chin of the subject when the EEG headgear is worn by the subject, a transverse strap portion secured to the coronal strap portion and extending transversely around a subject's head when the EEG headgear is worn by the subject, and a sagittal strap portion secured to the transverse strap portion and the coronal strap portion;
(b) a plurality of electrodes, each electrode comprising an electrode head having an upper side and a lower side and a wire having a proximal end and a distal end, wherein the electrode head is in electrical communication with the wire;
(c) a plurality of strap openings extending between the upper side and lower side of the one or more elastic straps; and
(d) a plurality of pocket fasteners formed from elastic panels, each elastic panel having a proximal side, distal side, right side, left side, upper side, and lower side, wherein each elastic panel is positioned over a strap opening of the one or more elastic straps of the EEG headgear so as to at least partially cover the strap opening, and wherein each elastic panel is secured to strap material adjacent to the strap opening on the upper side of the one or more elastic straps, each elastic panel being secured to the strap material along three sides, the three sides being selected from the group consisting of the proximal side, distal side, right side, and left side of the elastic panel, thereby forming a pocket fastener having a receptacle between the lower side of the elastic panel and the upper side of the strap material, the receptacle having a proximal opening,
wherein an electrode head can be placed into the receptacle of a pocket fastener through the proximal opening of the pocket fastener so as to place the lower side of the electrode head over or through the strap opening below the elastic panel of the pocket fastener in order to place the electrode in electrical contact with the scalp of the subject when the EEG headgear is worn by the subject, and
wherein the elastic panel is stretched and placed in tension when the electrode head is placed in the receptacle, and wherein the lower side of the elastic panel exerts a force on the upper side of the electrode head due to such tension, thereby retaining the electrode head within the pocket fastener and in contact with the scalp of the subject.

2. The headgear assembly of claim 1, wherein the plurality of strap openings are positioned according to the International 10-20 System.

3. The headgear assembly of claim 2, wherein the strap openings are positioned at least at points Fp1, Fp2, F7, F8, T3, T4, Cz, C3, C4, T5, T6, O1, and O2.

4. The headgear assembly of claim 1, wherein the electrode head comprises an electrode cup for retaining a conductive gel.

5. The headgear assembly of claim 1, wherein the electrode head is a disc electrode or a needle electrode.

6. The headgear assembly of claim 1, wherein the transverse strap portion, coronal strap portion, and sagittal strap portion are formed from separate straps.

7. The headgear assembly of claim 1, wherein the coronal strap portion comprises a reversibly securable chin strap.

8. The headgear assembly of claim 1, further comprising a bias strap secured to coronal strap portion and the transverse strap portion.

9. The headgear assembly of claim 1, wherein the elastic panel is secured to the strap material using thread.

10. The headgear assembly of claim 1, further comprising a wireless transmitter module in electrical communication with the plurality of electrodes for transmitting an electrical signal received by the electrodes from the subject's brain,

11. The headgear assembly of claim 1, further comprising a container for the wireless transmitter module attached to the sagittal strap portion at the crown of the subject's head.

12. The headgear assembly of claim 11, wherein the container is removably secured to the sagittal strap portion.

13. The headgear assembly of claim 1, wherein the wireless transmitter module comprises a processor for signal processing and/or an electrode impedance indicator.

14. (canceled)

15. A method of performing EEG, comprising the steps of positioning the headgear assembly of claim 1 on a subject's head and detecting electrical activity in the subject's brain.

16. The method of claim 15, further comprising the steps of:
positioning the EEG headgear of the headgear assembly on a subject's head; and
placing the electrode heads of the headgear assembly into the pocket fasteners.

17. The method of claim 15, further comprising the step of recording the electrical activity in the subject's brain.
